(19) United States
(12) Patent Application Publication
(10) Pub. No.: US 2024/0390654 A1
(43) Pub. Date: Nov. 28, 2024

(54) CATHETER SYSTEM HAVING A GUIDEWIRE AND SPRING-LOADED NEEDLE RETRACTION

(71) Applicant: Becton, Dickinson and Company, Franklin Lakes, NJ (US)

(72) Inventor: Clark Ragsdale, Herriman, UT (US)

(21) Appl. No.: 18/658,798

(22) Filed: May 8, 2024

Related U.S. Application Data
(60) Provisional application No. 63/468,947, filed on May 25, 2023.

(51) Int. Cl. A61M 25/06; A61M 25/00; A61M 25/09

(52) U.S. Cl. CPC A61M 25/065; A61M 25/0097; A61M 25/09

(57) ABSTRACT

A catheter system may include a catheter assembly, which may include a catheter hub and a catheter. The catheter system may include a barrel, a clip and needle hub disposed within the barrel, an introducer needle secured within the needle hub, and a catheter hub slider extending through a slot of the barrel and configured to slide along the slot from a catheter hub slider proximal position to a distally-advanced position. The catheter system may include guidewire configured to advance distally through the introducer needle. The catheter system may include a guidewire slider extending through the slot and configured to slide along the slot from a guidewire slider proximal position to a distal position to advance the guidewire. When the guidewire slider is in the guidewire slider proximal position, the catheter hub slider may be prevented from sliding along the slot.

### RELATED APPLICATIONS

This application claims the benefit of U.S. Provisional Patent Application No. 63/468,947, which was filed on May 25, 2023, which is incorporated herein in its entirety.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to a catheter system having a guidewire and spring-loaded needle retraction, as well as related devices and methods.

In some embodiments, the catheter system may include a catheter hub, which may include a distal end, a proximal end, and a lumen extending through the distal end of the catheter hub and the proximal end of the catheter hub. In some embodiments, the catheter system may include a catheter, which may include a distal tip. In some embodiments, the catheter may extend from the distal end of the catheter hub.

In some embodiments, the catheter system may include a barrel, which may include a distal end, a proximal end, and a slot. In some embodiments, the catheter system may include a flexible arm. In some embodiments, the catheter system may include a clip disposed within the barrel. In some embodiments, the catheter system may include a needle hub disposed within the barrel. In some embodiments, the catheter system may include an introducer needle, which may include a sharp distal tip and a proximal end. In some embodiments, the proximal end of the introducer needle may be secured within the needle hub. In some embodiments, the catheter system may include a spring coupled to the needle hub.

In some embodiments, the catheter system may include a catheter hub slider extending through the slot. In some embodiments, the catheter hub slider may include a distally-extending arm. In some embodiments, the catheter system may include a guidewire, which may include a first end and a second end. In some embodiments, the first end may be configured to advance distally through the introducer needle. In some embodiments, the catheter system may include a guidewire slider extending through the slot.

In some embodiments, the catheter system may be configured to move from a first position to a second position. In some embodiments, when the catheter system is in the first position, the guidewire slider may be disposed in the guidewire slider proximal position and configured to slide along the slot from the guidewire slider proximal position to a distal position to advance the guidewire. In some embodiments, when the catheter system is in the first position, the catheter hub slider may be disposed in a catheter hub slider proximal position. In some embodiments, when the catheter system is in the first position, the clip may couple the catheter hub to the barrel to secure the catheter hub with respect to the barrel. In some embodiments, when the catheter system is in the first position, the distally-extending arm of the catheter hub slider may be coupled to the catheter hub to prevent distal movement and proximal movement of the catheter hub slider with respect to the catheter hub. In some embodiments, when the catheter system is in the first position, the introducer needle may extend through the catheter. In some embodiments, when the catheter system is in the first position, the flexible arm may contact the needle hub to maintain the spring in a compressed position.

In some embodiments, when the catheter system is in the second position, the guidewire slider may be disposed in the distal position such that the guidewire is advanced beyond the distal tip of the catheter. In some embodiments, when the catheter system is in the second position, the catheter hub slider may be disposed in the catheter hub slider proximal position. In some embodiments, when the catheter system is in the second position, the distally-extending arm of the catheter hub slider may be coupled to the catheter hub to prevent distal movement and proximal movement of the catheter hub slider with respect to the catheter hub. In some embodiments, when the catheter system is in the second position, the introducer needle may extend through the catheter. In some embodiments, when the catheter system is in the second position, the flexible arm may contact the needle hub to maintain the spring in the compressed position.

In some embodiments, in response to movement of the catheter system from the first position to the second position, an elongated arm of the guidewire slider may contact the clip to uncouple the clip from the catheter hub. In some embodiments, in response to the clip uncoupling from the catheter hub, the catheter hub slider may be configured to slide from the catheter hub slider proximal position to a distally-advanced position. In some embodiments, in response to sliding the catheter hub slider from the catheter hub slider proximal position to the distally-advanced position, the catheter hub may be configured to be distally advanced.

In some embodiments, the second end of the guidewire may be fixed within the barrel. In some embodiments, the guidewire slider may include an arced slot therethrough. In some embodiments, when the catheter system is in the first position, the guidewire may be disposed in the arced slot in a double-throw configuration such that there is a 2:1 advancement ratio between the first end of the guidewire and the guidewire slider. In some embodiments, the arced slot may be oriented horizontally within the barrel. In some embodiments, the barrel may include a proximal inner ramp. In some embodiments, in response to movement of the catheter system from the first position to the second position, the guidewire slider may be lifted within the barrel by the proximal inner ramp such that the guidewire is removed from the arced slot.

In some embodiments, the catheter hub may include a groove. In some embodiments, the barrel may include a distal inner ramp. In some embodiments, the catheter system may be configured to move from the second position to a third position. In some embodiments, when the catheter system is in the third position, the guidewire slider may be disposed in the distal position such that the guidewire is advanced beyond the distal tip of the catheter. In some embodiments, when the catheter system is in the third position, the catheter hub slider may be disposed in the distally-advanced position. In some embodiments, when the catheter system is in the third position, the introducer needle may extend through the catheter. In some embodiments, when the catheter system is in the third position, the flexible arm may contact the needle hub to maintain the spring in the compressed position.

In some embodiments, in response to movement of the catheter system from the second position to the third position, the distally-extending arm of the catheter hub slider may contact the distal inner ramp of the barrel to lift the distally-extending arm from the groove of the catheter hub to uncouple the catheter hub slider from the catheter hub. In some embodiments, the proximal end of the catheter hub may include the groove.

In some embodiments, the distally-advanced position of the catheter hub slider is a partially distally-advanced position. In some embodiments, the catheter system may be configured to move from the third position to a fourth position. In some embodiments, when the catheter system is in the fourth position, the guidewire slider is disposed in the distal position such that the guidewire is advanced beyond the distal tip of the catheter. In some embodiments, when the catheter system is in the fourth position, the catheter hub slider may be disposed in a fully distally-advanced position. In some embodiments, in response to movement of the catheter system from the third position to the fourth position, the catheter hub slider may push out on the flexible arm such that the flexible arm is removed from contact with the needle hub and the spring expands to move the needle hub proximally and retract the introducer needle into the barrel.

In some embodiments, the flexible arm may include a fin tapered inwardly in a distal direction. In some embodiments, in response to movement of the catheter system from the third position to the fourth position, the catheter hub slider may push out on the fin such that the flexible arm is removed from contact with the needle hub and the spring expands to move the needle hub proximally and retract the introducer needle into the barrel.

In some embodiments, the flexible arm is a first flexible arm. In some embodiments, the barrel may include a second flexible arm proximal to the first flexible arm. In some embodiments, the second flexible arm may include a second fin tapered inwardly in a proximal direction. In some embodiments, in response to the spring expanding to move the needle hub proximally, the second flexible arm may be biased outwardly to allow the needle hub to pass the second fin and then snap inwardly to trap the needle hub proximal to the second fin. In some embodiments, when the catheter system is between the first position and the second position, the guidewire slider is configured to move back and forth or distally and proximally.

In some embodiments, when the catheter system is in the first position, the guidewire slider may be on top of the catheter hub slider. In some embodiments, a proximal end of the catheter hub slider may include a tab to prevent the guidewire slider from sliding proximal to the tab. In some embodiments, the distally-extending arm of the catheter hub slider may include a hook and/or an inward projection proximal to the hook. In some embodiments, in response to movement of the catheter system from the second position to the third position, the distally-extending arm of the catheter hub slider may contact the distal inner ramp on the inner surface of the barrel to lift the hook from the groove of the catheter hub to uncouple the catheter hub slider from the catheter hub, wherein when the hook is lifted from the groove the inward projection remains in contact with the proximal end of the catheter hub such that the catheter hub slider is configured to push the catheter hub in a distal direction.

In some embodiments, when the guidewire slider is in the guidewire slider proximal position, the catheter hub slider may be prevented from sliding along the slot from the catheter hub slider proximal position to the distally-advanced position. In some embodiments, the spring may be configured to expand and retract the introducer needle into the barrel only after the catheter hub slider advances the catheter hub distally and is uncoupled from the catheter hub.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example catheter system in an example first position, according to some embodiments;

FIG. 1B is a cross-sectional view of the catheter system along line 1B-1B of FIG. 1A, according to some embodiments;

FIG. 1C is a cross-sectional view of the catheter system along line 1C-1C of FIG. 1A, according to some embodiments;

FIG. 1D is another cross-sectional view of the catheter system, according to some embodiments;

FIG. 2A is an upper perspective view of the catheter system in an example second position, according to some embodiments;

FIG. 2B is an upper perspective view of the catheter system in the second position, illustrating an example clip uncoupled from an example catheter hub, according to some embodiments;

FIG. 2C is a partial cutaway view of the catheter system in the second position, illustrating an example guidewire slider lifted within an example barrel by an example proximal inner ramp such that an example guidewire may be removed from an example arced slot;

FIG. 2D is a partial cutaway view of the catheter system in the second position, illustrating an example needle hub locked in place by an example fin, according to some embodiments;

FIG. 2E is a cross-sectional view of the catheter system along line 2E-2E of FIG. 2A, according to some embodiments;

FIG. 2F is a cross-sectional view of the catheter system along line 2F-2F of FIG. 2A, according to some embodiments;

FIG. 2G is a partial cutaway view of the catheter system being moved from the second position to an example third position and an example catheter hub slider configured to push and pull an example catheter hub, according to some embodiments;

FIG. 3A is a top view of the catheter system in the third position, according to some embodiments;

FIG. 3B is a cross-sectional view of the catheter system along line 3B-3B of FIG. 3A, according to some embodiments;

FIG. 3C is a cross-sectional view of the catheter system along line 3C-3C of FIG. 3A, according to some embodiments;

FIG. 4A is a top view of the catheter system in an example fourth position, according to some embodiments;

FIG. 4B is a cross-sectional view of the catheter system along line 4B-4B of FIG. 4A, according to some embodiments;

FIG. 4C is a cross-sectional view of the catheter system along line 4C-4C of FIG. 4A, according to some embodiments;

FIG. 4D is an upper perspective view of the catheter system being moved from the third position to the fourth position and the catheter hub slider pushing out on the flexible arm to remove the flexible arm from contact with the needle hub; and

FIG. 4E is a partial cutaway view of the catheter system, illustrating the needle hub locked in the proximal end of the barrel, according to some embodiments.

### DESCRIPTION OF EMBODIMENTS

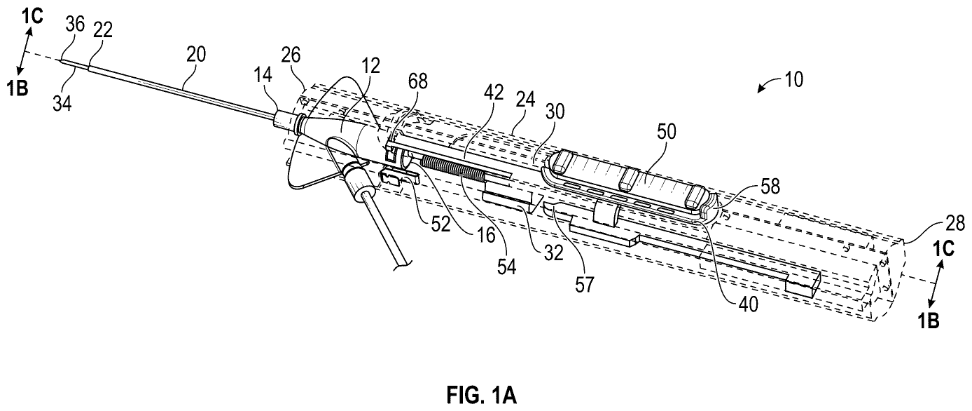

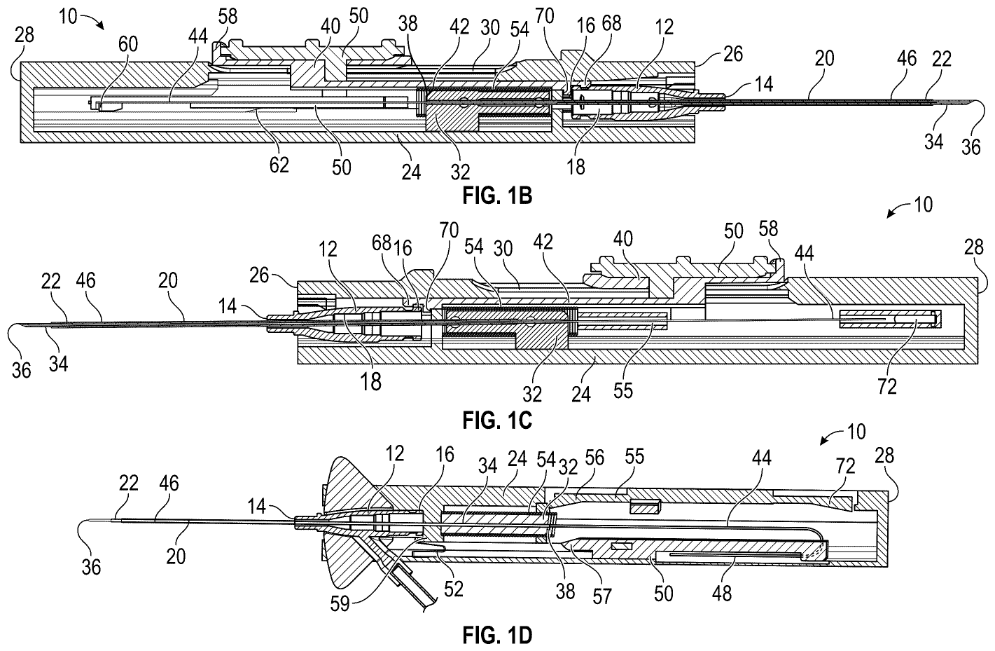

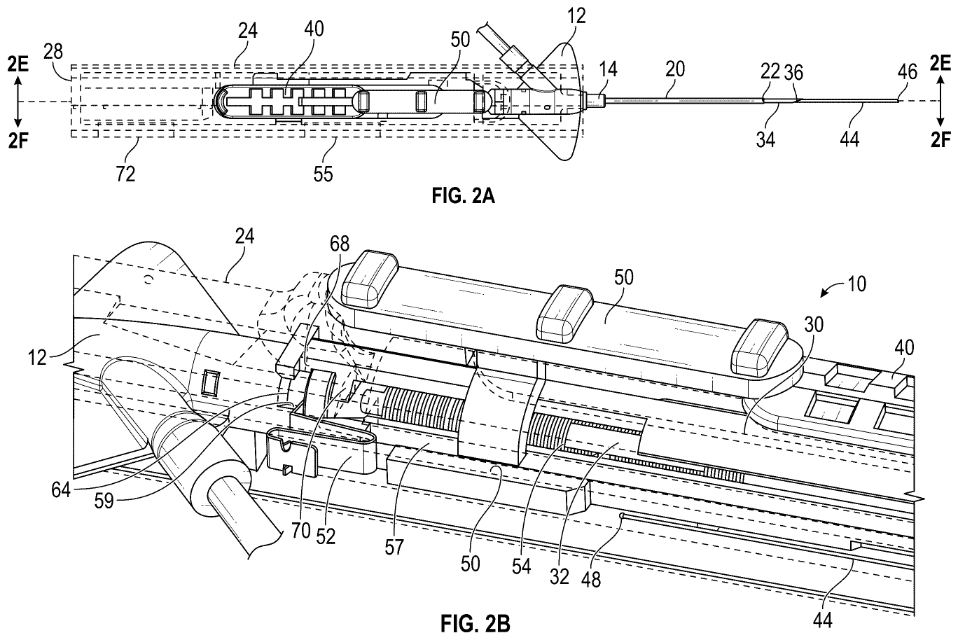

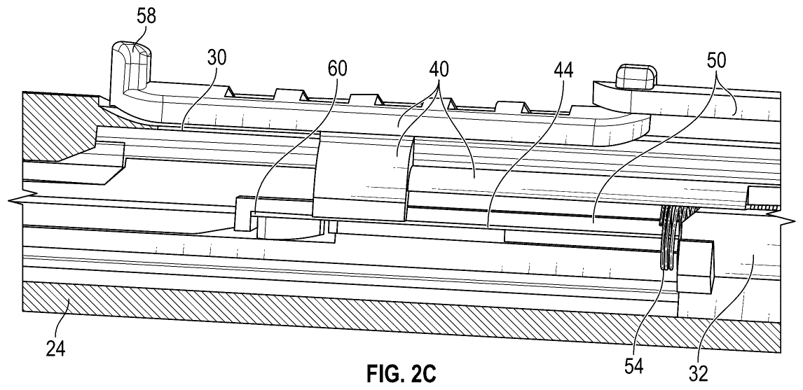

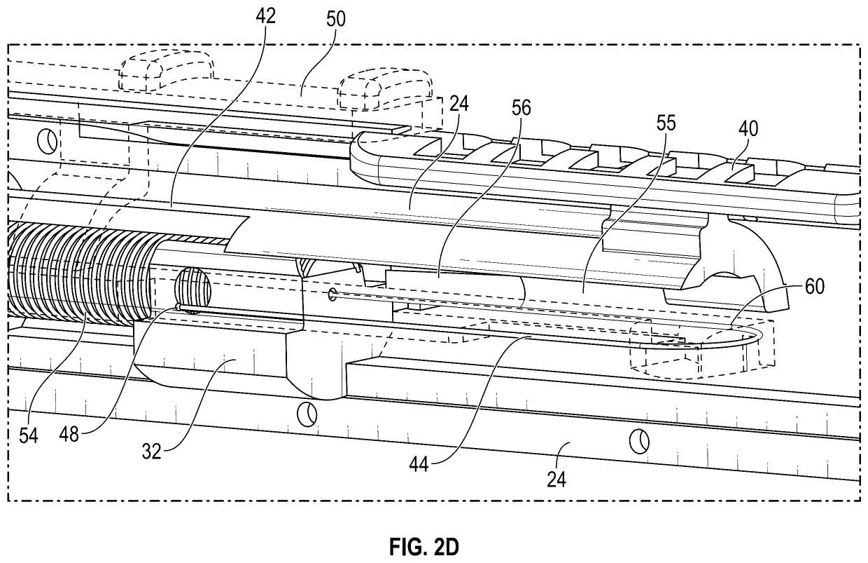

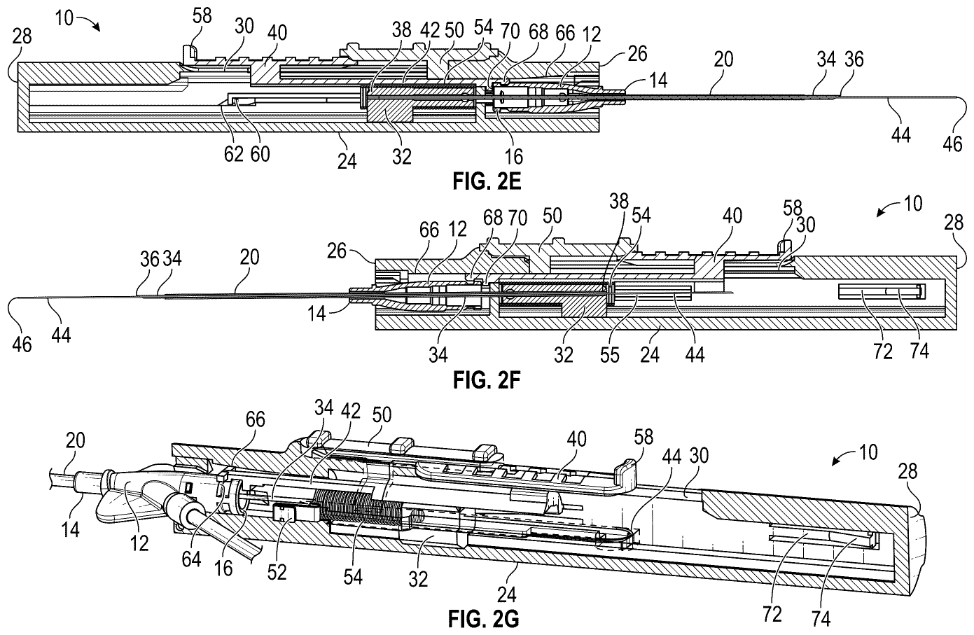

Referring now to FIGS. 1-2, in some embodiments, a catheter system 10 may include a catheter hub 12, which may include a distal end 14, a proximal end 16, and a lumen 18 extending through the distal end 14 of the catheter hub 12 and the proximal end 16 of the catheter hub 12. In some embodiments, the catheter system 10 may include a catheter 20, which may include a distal tip 22. In some embodiments, the catheter 20 may extend from the distal end 14 of the catheter hub 12. In some embodiments, the catheter system 10 may include an arterial catheter system, a peripheral intravenous catheter system, a midline catheter system, a peripherally-inserted central catheter system, or another suitable catheter system.

In some embodiments, the catheter system 10 may include a barrel 24, which may include a distal end 26, a proximal end 28, and a slot 30. In some embodiments, the catheter system 10 may include a needle hub 32 disposed within the barrel 24. In some embodiments, the catheter system 10 may include an introducer needle 34, which may include a sharp distal tip 36 and a proximal end 38. In some embodiments, the proximal end 38 of the introducer needle 34 may be secured within the needle hub 32.

In some embodiments, the catheter system 10 may include a catheter hub slider 40 extending through the slot 30 and configured to move along the slot 30. In some embodiments, the catheter hub slider 40 may include a distally-extending arm 42 configured to couple to the catheter hub 12. In some embodiments, the catheter system 10 may include a guidewire 44, which may include a first end 46 and a second end 48. In some embodiments, the first end 46 may be configured to advance distally through the introducer needle 34. In some embodiments, the catheter system 10 may include a guidewire slider 50 extending through the slot 30 and configured to move along the slot 30.

In some embodiments, the catheter system 10 may be configured to move from a first position, illustrated for example in FIGS. 1A-1E, to a second position, illustrated for example in FIGS. 2A-2E. In some embodiments, when the catheter system 10 is in the first position, the guidewire slider 50 may be disposed in the guidewire slider proximal position and configured to slide along the slot 30 from the guidewire slider proximal position to a distal position to advance the guidewire 44. In some embodiments, when the catheter system 10 is between the first position and the second position, the guidewire slider 50 may be configured to move back and forth to change a position of the first end 46 of the guidewire 44 with respect to the catheter 20.

In some embodiments, when the catheter system 10 is in the first position, the catheter hub slider 40 may be disposed in a catheter hub slider proximal position. In some embodiments, when the guidewire slider 50 is in the guidewire slider proximal position, the catheter hub slider 40 may be prevented from sliding along the slot 30. In further detail, in some embodiments, when the guidewire slider 50 is in the guidewire slider proximal position, the catheter hub slider 40 may be prevented from sliding from the catheter hub slider proximal position to a distally-advanced position. Thus, in some embodiments, distal advancement of the catheter hub 12 is prevented until after the guidewire 44 is advanced distally.

In some embodiments, the catheter system 10 may include a clip 52. In some embodiments, when the catheter system 10 is in the first position, the clip 52 may couple the catheter hub 12 to the barrel 24 to secure the catheter hub 12 with respect to the barrel 24. In some embodiments, when the catheter system 10 is in the first position, the distally-extending arm 42 of the catheter hub slider 40 may be coupled to the catheter hub 12 to prevent distal movement and proximal movement of the catheter hub slider 40 with respect to the catheter hub 12. In some embodiments, when the catheter system 10 is in the first position, the introducer needle 34 may extend through the catheter 20. In some embodiments, the catheter system 10 may be ready for insertion into a blood vessel of a patient in the first position.

In some embodiments, the catheter system 10 may include a spring 54, which may be coupled to the needle hub 32. In some embodiments, the spring 54 may act as a passive safety mechanism to facilitate retraction of the introducer needle 34 into the barrel 24, as will be explained later in further detail. In some embodiments, the catheter system 10 may include a flexible arm 55. In some embodiments, the barrel 24 may include the flexible arm 55. In some embodiments, when the catheter system 10 is in the first position, the flexible arm 55 may contact the needle hub 32 to maintain the spring 54 in a compressed position. In some embodiments, the flexible arm 55 may include a fin 56 tapered inwardly in a distal direction. In some embodiments, when the catheter system 10 is in the first position, the fin 56 may contact the needle hub 32 to maintain the spring 54 in a compressed position.

In some embodiments, when the catheter system 10 is in the first position, the guidewire slider 50 may be on top of the catheter hub slider 40. In some embodiments, given the catheter hub slider 40 may not be slid along the slot 30 or moved until after the guidewire slider 50 is moved, the guidewire slider 50 on top of the catheter hub slider 40 may facilitate a compact, ergonomically-friendly device without negatively impacting use of the catheter hub slider 40 by the clinician. In some embodiments, a proximal end of the catheter hub slider 40 may include a tab 58 to prevent the guidewire slider 50 from sliding proximal to the tab 58.

In some embodiments, when the catheter system 10 is in the second position, the guidewire slider 50 may be disposed in the distal position such that the guidewire 44 may be advanced beyond the distal tip 22 of the catheter 20. In some embodiments, the first end 46 of the guidewire 44 may be disposed within the introducer needle 34 in the first position and may exit the sharp distal tip 36 of the introducer needle 34 in response to movement of the catheter system 10 from the first position to the second position.

In some embodiments, when the catheter system 10 is in the second position, the catheter hub slider 40 may be disposed in the catheter hub slider proximal position. In some embodiments, when the catheter system 10 is in the second position, the distally-extending arm 42 of the catheter hub slider 40 may be coupled to the catheter hub 12 to prevent distal movement and proximal movement of the catheter hub slider 40 with respect to the catheter hub 12. In some embodiments, when the catheter system 10 is in the second position, the introducer needle 34 may extend through the catheter 20. In some embodiments, when the catheter system 10 is in the second position, the fin 56 may contact the needle hub 32 to maintain the spring in the compressed position.

In some embodiments, in response to movement of the catheter system 10 from the first position to the second position, an elongated arm 57 of the guidewire slider 50 may contact the clip 52 to uncouple the clip 52 from the catheter hub 12. As illustrated for example in FIG. 2B, in some embodiments, the clip 52 may include a V-shaped clip with a pawl 59 inserted into a groove 64 of the catheter hub 12 in the first position. In some embodiments, when the catheter system 10 moves from the first position to the second position and the guidewire slider 50 is advanced distally, the elongated arm 57 may compress the V-shaped clip, removing the pawl 59 from the groove 64. In some embodiments, in response to the clip 52 uncoupling from the catheter hub 12, the catheter hub slider 40 may be configured to slide from the catheter hub slider proximal position to the distally-advanced position. In some embodiments, in response to sliding the catheter hub slider 40 from the catheter hub slider proximal position to the distally-advanced position, the catheter hub 12 may be configured for distal advancement.

In some embodiments, the second end 48 of the guidewire 44 may be fixed within the barrel 24. In some embodiments, the guidewire slider 50 may include an arced slot 60 therethrough, facilitating movement of the guidewire 44 therethrough in a double throw configuration. In some embodiments, when the catheter system 10 is in the first position, the guidewire 44 may be disposed in the arced slot 60 in the double-throw configuration such that there is a 2:1 advancement ratio between the first end 46 of the guidewire 44 and the guidewire slider 50. In some embodiments, the barrel 24 may include a proximal inner ramp 62. In some embodiments, in response to movement of the catheter system 10 from the first position to the second position, the guidewire slider 50 may be lifted within the barrel 24 by the proximal inner ramp 62 such that the guidewire 44 is removed from the arced slot 60, as illustrated for example in FIG. 2C. In some embodiments, the arced slot 60 may be oriented horizontally within the barrel, which may facilitate removal of the guidewire 44 from the arced slot 60 by gravity when a portion of the guidewire slider 50 including the arced slot 60 is lifted in response to contact with the proximal inner ramp 62. In some embodiments, when the catheter system 10 is between the first position and the second position, the guidewire slider 50 may be configured to move back and forth between the guidewire slider proximal position and the distal position. In some embodiments, after the guidewire slider 50 has been moved to the distal position, the guidewire 44 may no longer be retracted with respect to the introducer needle 34 because the guidewire 44 has dropped out of the arced slot 60.

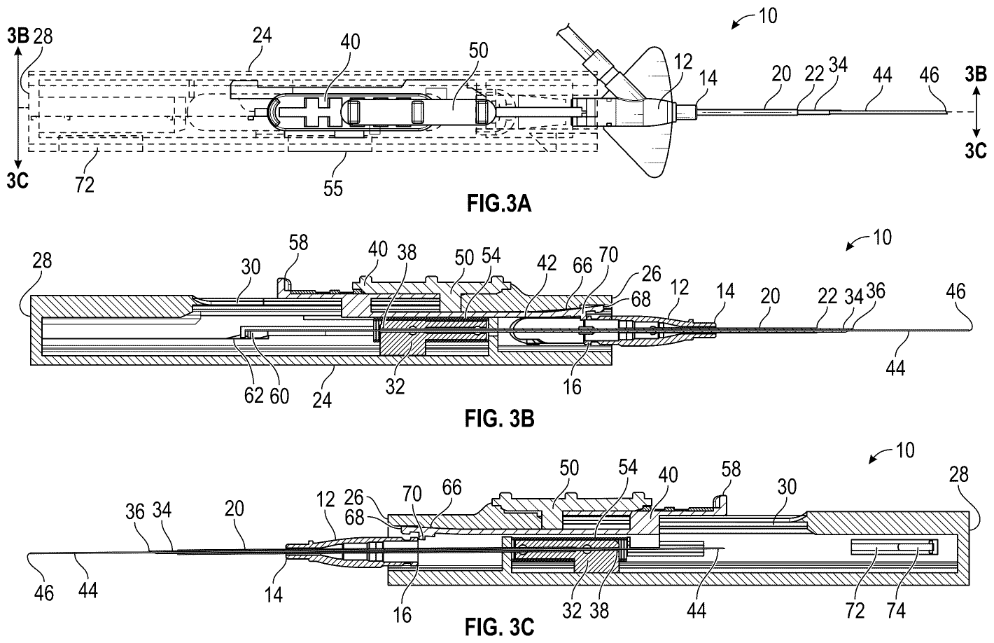

Referring now to FIGS. 3A-3C, in some embodiments, the barrel 24 may include a distal inner ramp 66. In some embodiments, the catheter system 10 may be configured to move from the second position to a third position, illustrated for example in FIGS. 3A-3C. In some embodiments, when the catheter 20 system is in the third position, the guidewire slider 50 may be disposed in the distal position such that the guidewire 44 is advanced beyond the distal tip 22 of the catheter 20. In some embodiments, when the catheter system 10 is in the third position, the catheter hub slider 40 may be disposed in the distally-advanced position. In some embodiments, when the catheter system 10 is in the third position, the introducer needle 34 may extend through the catheter 20. In some embodiments, when the catheter system 10 is in the third position, the flexible arm 55 and/or the fin 56 may contact the needle hub 32 to maintain the spring 54 in the compressed position. Thus, in some embodiments, the introducer needle 34 may be prevented from being retracted or proximally withdrawn before the guidewire 44 and the catheter hub 12 are distally advanced.

In some embodiments, in response to movement of the catheter system 10 from the first position to the second position, the distally-extending arm 42 of the catheter hub slider 40 may contact the distal inner ramp 66 of the barrel 24 to lift the distally-extending arm 42 from the groove 64 of the catheter hub 12 to uncouple the catheter hub slider 40 from the catheter hub 12. In some embodiments, the proximal end 16 of the catheter hub 12 may include the groove 64.

In some embodiments, the distally-extending arm 42 of the catheter hub slider 40 may include a hook 68 and/or an inward projection 70 proximal to the hook 68. In some embodiments, in response to movement of the catheter system 10 from the second position to the third position, the distally-extending arm 42 of the catheter hub slider 40 may contact the distal inner ramp 66 on the inner surface of the barrel 24 to lift the hook 68 from the groove 64 of the catheter hub 12 to uncouple the catheter hub slider 40 from the catheter hub 12. In some embodiments, when the hook 68 is lifted from the groove 64 the inward projection 70 may remain in contact with the proximal end 16 of the catheter hub 12 such that the catheter hub slider 40 is still configured to push the catheter hub 12 in a distal direction.

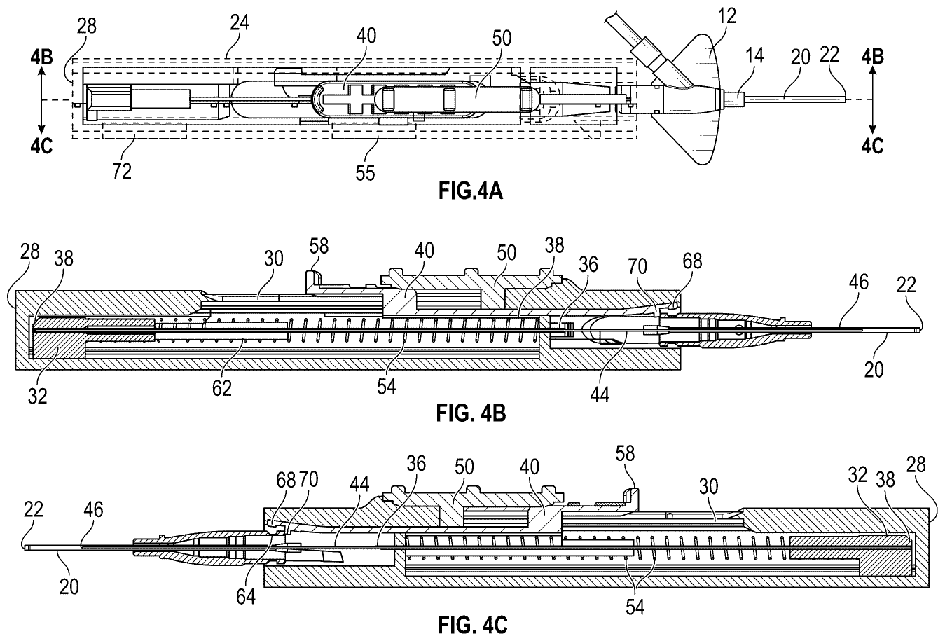

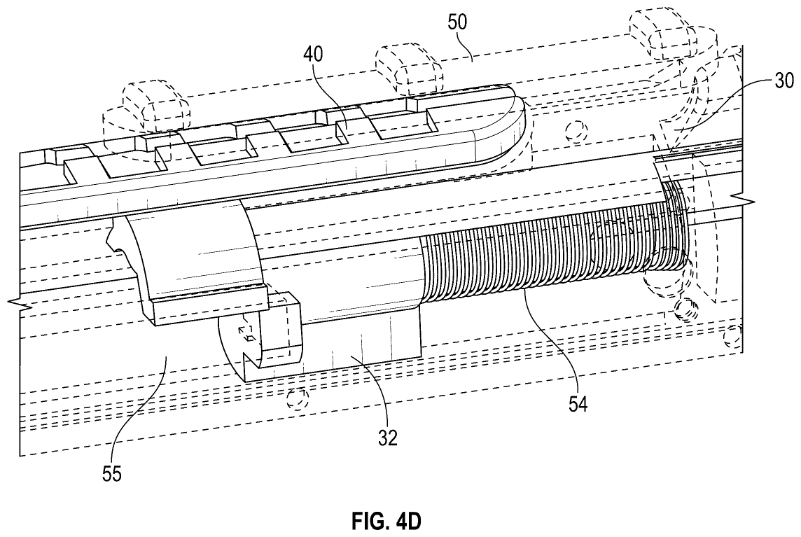

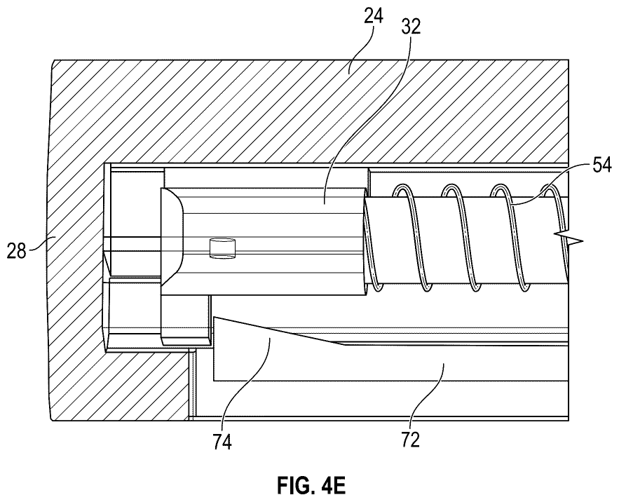

Referring now to FIGS. 4A-4E, in some embodiments, the distally-advanced position of the catheter hub slider 40 is a partially distally-advanced position. In some embodiments, the catheter system 10 may be configured to move from the third position to a fourth position, illustrated for example in FIGS. 4A-4D. In some embodiments, when the catheter system 10 is in the fourth position, the guidewire slider 50 is disposed in the distal position such that the guidewire 44 is advanced beyond the distal tip 22 of the catheter 20. In some embodiments, when the catheter system 10 is in the fourth position, the catheter hub slider 40 may be disposed in a fully distally-advanced position. In some embodiments, in response to movement of the catheter system 10 from the third position to the fourth position, the catheter hub slider 40 may push out on the flexible arm 55 such that the flexible arm 55 and/or the fin 56 is removed from contact with the needle hub 32 and the spring 54 expands to move the needle hub 32 proximally and retract the introducer needle 34 into the barrel 24.

In some embodiments, the spring 54 may be configured to expand and retract the introducer needle 34 into the barrel 24 only after the catheter hub slider 40 advances the catheter hub 12 distally and is uncoupled from the catheter hub 12, thus preventing premature retraction of the introducer needle 34. In some embodiments, the flexible arm 55 is a first flexible arm. In some embodiments, the barrel 24 may include a second flexible arm 72 proximal to the first flexible arm. In some embodiments, the second flexible arm 72 may include a second fin 74 tapered inwardly in a proximal direction. In some embodiments, in response to the spring 54 expanding to move the needle hub 32 proximally, the second flexible arm 72 may be biased outwardly to allow the needle hub 32 to pass the second fin 74 and then snap inwardly to trap the needle hub 32 proximal to the second fin 74, as illustrated for example in FIG. 4E.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter system, comprising:
a catheter assembly, comprising:
a catheter hub, comprising a distal end, a proximal end, and a lumen extending through the distal end of the catheter hub and the proximal end of the catheter hub; and
a catheter comprising a distal tip and extending from the distal end of the catheter hub;
a barrel, comprising a distal end, a proximal end, a slot, a flexible arm;
a clip disposed within the barrel;
a needle hub disposed within the barrel;
an introducer needle comprising a sharp distal tip and a proximal end, wherein the proximal end of the introducer needle is secured within the needle hub;
a spring coupled to the needle hub;
a catheter hub slider extending through the slot, wherein the catheter hub slider comprises a distally-extending arm;
a guidewire comprising a first end and a second end, wherein the first end is configured to advance distally through the introducer needle; and
a guidewire slider extending through the slot and comprising an elongated arm,
wherein the catheter system is configured to move from a first position to a second position, wherein when the catheter system is in the first position:
the guidewire slider is disposed in the guidewire slider proximal position and configured to slide along the slot from the guidewire slider proximal position to a distal position to advance the guidewire;
the catheter hub slider is disposed in a catheter hub slider proximal position;
the clip couples the catheter hub to the barrel to secure the catheter hub with respect to the barrel;
the distally-extending arm of the catheter hub slider is coupled to the catheter hub to prevent distal movement and proximal movement of the catheter hub slider with respect to the catheter hub;
the introducer needle extends through the catheter; and
the flexible arm contacts the needle hub to maintain the spring in a compressed position;
wherein when the catheter system is in the second position:
the guidewire slider is disposed in the distal position such that the guidewire is advanced beyond the distal tip of the catheter;
the catheter hub slider is disposed in the catheter hub slider proximal position;
the distally-extending arm of the catheter hub slider is coupled to the catheter hub to prevent distal movement and proximal movement of the catheter hub slider with respect to the catheter hub;
the introducer needle extends through the catheter; and
the flexible arm contacts the needle hub to maintain the spring in the compressed position,
wherein in response to movement of the catheter system from the first position to the second position the elongated arm of the guidewire slider contacts the clip to uncouple the clip from the catheter hub, wherein in response to the clip uncoupling from the catheter hub, the catheter hub slider is configured to slide from the catheter hub slider proximal position to a distally-advanced position, wherein in response to sliding the catheter hub slider from the catheter hub slider proximal position to the distally-advanced position, the catheter hub may be configured to be distally advanced.

2. The catheter system of claim 1, wherein the second end of the guidewire is fixed within the barrel, wherein the guidewire slider comprises an arced slot therethrough, wherein when the catheter system is in the first position, the guidewire is disposed in the arced slot in a double-throw configuration such that there is a 2:1 advancement ratio between the first end of the guidewire and the guidewire slider.

3. The catheter system of claim 2, wherein the arced slot is oriented horizontally within the barrel.

4. The catheter system of claim 2, wherein the barrel comprises a proximal inner ramp, wherein in response to movement of the catheter system from the first position to the second position, the guidewire slider is lifted within the barrel by the proximal inner ramp such that the guidewire is removed from the arced slot.

5. The catheter system of claim 1, wherein the catheter hub comprises a groove, wherein the barrel further comprises a distal inner ramp, wherein the catheter system is configured to move from the second position to a third position, wherein when the catheter system is in the third position:
the guidewire slider is disposed in the distal position such that the guidewire is advanced beyond the distal tip of the catheter;
the catheter hub slider is disposed in the distally-advanced position;
the introducer needle extends through the catheter; and
the flexible arm contacts the needle hub to maintain the spring in the compressed position,
wherein in response to movement of the catheter system from the second position to the third position, the distally-extending arm of the catheter hub slider contacts the distal inner ramp of the barrel to lift the distally-extending arm from the groove of the catheter hub to uncouple the catheter hub slider from the catheter hub.

6. The catheter system of claim 5, wherein the barrel further comprises a distal inner ramp, wherein the distally-extending arm of the catheter hub slider comprises a hook and an inward projection proximal to the hook, wherein in response to movement of the catheter system from the second position to the third position, the distally-extending arm of the catheter hub slider contacts the distal inner ramp on the inner surface of the barrel to lift the hook from the groove of the catheter hub to uncouple the catheter hub slider from the catheter hub, wherein when the hook is lifted from the groove the inward projection remains in contact with the proximal end of the catheter hub such that the catheter hub slider is configured to push the catheter hub in a distal direction.

7. The catheter system of claim 5, wherein the proximal end of the catheter hub comprises a groove, wherein the distally-advanced position of the catheter hub slider is a partially distally-advanced position, wherein the catheter system is configured to move from the third position to a fourth position, wherein when the catheter system is in the fourth position:
the guidewire slider is disposed in the distal position such that the guidewire is advanced beyond the distal tip of the catheter; and
the catheter hub slider is disposed in a fully distally-advanced position, wherein in response to movement of the catheter system from the third position to the fourth position the catheter hub slider pushes out on the flexible arm such that the flexible arm is removed from contact with the needle hub and the spring expands to move the needle hub proximally and retract the introducer needle into the barrel.

8. The catheter system of claim 7, wherein the flexible arm comprises a fin tapered inwardly in a distal direction, wherein in response to movement of the catheter system from the third position to the fourth position the catheter hub slider pushes out on the fin such that the flexible arm is removed from contact with the needle hub and the spring expands to move the needle hub proximally and retract the introducer needle into the barrel.

9. The catheter system of claim 1, wherein the flexible arm is a first flexible arm, wherein the barrel comprises a second flexible arm proximal to the first flexible arm and comprising a second fin tapered inwardly in a proximal direction, wherein in response to the spring expanding to move the needle hub proximally, the second flexible arm is biased outwardly to allow the needle hub to pass the second fin and then snaps inwardly to trap the needle hub proximal to the second fin.

10. The catheter system of claim 1, wherein when the catheter system is between the first position and the second position, the guidewire slider is configured to move back and forth.

11. The catheter system of claim 1, wherein when the catheter system is in the first position, the guidewire slider is on top of the catheter hub slider.

12. The catheter system of claim 11, wherein a proximal end of the catheter hub slider comprises a tab to prevent the guidewire slider from sliding proximal to the tab.

13. A catheter system, comprising:
a catheter assembly, comprising:
a catheter hub, comprising a distal end, a proximal end, and a lumen extending through the distal end of the catheter hub and the proximal end of the catheter hub; and
a catheter comprising a distal tip and extending from the distal end of the catheter hub;
a barrel, comprising a distal end, a proximal end, a slot;
a clip disposed within the barrel;
a needle hub disposed within the barrel;
an introducer needle comprising a sharp distal tip and a proximal end, wherein the proximal end of the introducer needle is secured within the needle hub;
a catheter hub slider extending through the slot and configured to slide along the slot from a catheter hub slider proximal position to a distally-advanced position;
a guidewire comprising a first end and a second end, wherein the first end is configured to advance distally through the introducer needle; and
a guidewire slider extending through the slot and configured to slide along the slot from a guidewire slider proximal position to a distal position to advance the guidewire, wherein when the guidewire slider is in the guidewire slider proximal position the catheter hub slider is prevented from sliding along the slot from the catheter hub slider proximal position to the distally-advanced position.

14. The catheter system of claim 13, wherein the catheter assembly further comprises a spring coupled to the needle hub, wherein the barrel further comprises a flexible arm, wherein when the catheter system is in a first position, the flexible arm contacts the needle hub to maintain the spring in a compressed position, wherein the spring is configured to expand and retract the introducer needle into the barrel only after the catheter hub slider advances the catheter hub distally and is uncoupled from the catheter hub.

15. The catheter system of claim 14, wherein the catheter hub slider comprises a distally-extending arm, wherein the catheter system is configured to move from the first position to a second position, wherein when the catheter system is in the first position:
the guidewire slider is disposed in the guidewire slider proximal position;
the catheter hub slider is disposed in the catheter hub slider proximal position;
the clip couples the catheter hub to the barrel to secure the catheter hub with respect to the barrel;
the distally-extending arm of the catheter hub slider is coupled to the catheter hub to prevent distal movement and proximal movement of the catheter hub slider with respect to the catheter hub; and
the introducer needle extends through the catheter;
wherein when the catheter system is in the second position,
the guidewire slider is disposed in the distal position such that the guidewire is advanced beyond the distal tip of the catheter;
the catheter hub slider is disposed in the catheter hub slider proximal position;
the distally-extending arm of the catheter hub slider is coupled to the catheter hub to prevent distal movement and proximal movement of the catheter hub slider with respect to the catheter hub;
the introducer needle extends through the catheter; and
the flexible arm contacts the needle hub to maintain the spring in the compressed position,
wherein in response to movement of the catheter system from the first position to the second position the distally-extending arm of the guidewire slider contacts the clip to uncouple the clip from the catheter hub, wherein in response to the clip uncoupling from the catheter hub, the catheter hub slider is configured to slide from the catheter hub slider proximal position to the distally-advanced position, wherein in response to sliding the catheter hub slider from the catheter hub slider proximal position to the distally-advanced position, the catheter hub may be configured to be distally advanced.

16. The catheter system of claim 13, wherein the second end of the guidewire is fixed within the barrel, wherein the guidewire slider comprises an arced slot therethrough, wherein when the catheter system is in the first position, the guidewire is disposed in the arced slot in a double-throw configuration such that there is a 2:1 advancement ratio between the first end of the guidewire and the guidewire slider.

17. The catheter system of claim 16, wherein the arced slot is oriented horizontally within the barrel.

18. The catheter system of claim 13, wherein the barrel comprises a proximal inner ramp, wherein in response to movement of the catheter system from the first position to the second position, the guidewire slider is lifted within the barrel by the proximal inner ramp such that the guidewire is removed from the arced slot.

19. The catheter system of claim 13, wherein the catheter hub slider comprises a distally-extending arm, wherein the catheter hub comprises a groove, wherein the barrel further comprises a distal inner ramp, wherein the catheter system is configured to move from the second position to a third position, wherein when the catheter system is in the third position:
the guidewire slider is disposed in the distal position such that the guidewire is advanced beyond the distal tip of the catheter;
the catheter hub slider is disposed in the distally-advanced position;
the introducer needle extends through the catheter; and
the flexible arm contacts the needle hub to maintain the spring in the compressed position,
wherein in response to movement of the catheter system from the second position to the third position, the distally-extending arm of the catheter hub slider contacts the distal inner ramp of the barrel to lift the distally-extending arm from the groove of the catheter hub to uncouple the catheter hub slider from the catheter hub.

20. The catheter system of claim 19, wherein the proximal end of the catheter hub comprises a groove, wherein the distally-advanced position of the catheter hub slider is a partially distally-advanced position, wherein the catheter system is configured to move from the third position to a fourth position, wherein when the catheter system is in the fourth position:
the guidewire slider is disposed in the distal position such that the guidewire is advanced beyond the distal tip of the catheter, and
the catheter hub slider is disposed in a fully distally-advanced position,
wherein in response to movement of the catheter system from the third position to the fourth position the catheter hub slider pushes out on the flexible arm such that the flexible arm is removed from contact with the needle hub and the spring expands to move the needle hub proximally and retract the introducer needle into the barrel.
